(19) United States
(12) Patent Application Publication
(10) Pub. No.: US 2022/0010800 A1
(43) Pub. Date: Jan. 13, 2022

(54) ENRICHMENT AND PURIFICATION DEVICE FOR SINGLE GAS IN MIXED GAS

(71) Applicant: University of Science and Technology of China, Hefei City (CN)

(72) Inventor: Bing Gong, Hefei City (CN)

(21) Appl. No.: 17/485,820

(22) Filed: Sep. 27, 2021

Related U.S. Application Data
(63) Continuation of application No. PCT/CN2020/073416, filed on Jan. 21, 2020.

(30) Foreign Application Priority Data
May 8, 2019 (CN) 2019103791163

(51) Int. Cl. F04D 19/04; F04B 37/08

(52) U.S. Cl. CPC F04D 19/042; B01D 8/00; F04B 37/08

(57) ABSTRACT

A device for enriching and purifying a single gas in a mixed gas includes: a pre-purification cold trap for freezing and adsorbing part of non-target gases; an enrichment cold and hot trap, having molecular sieves of different specifications detachably filled therein, a gas inlet thereof connected to a gas outlet of the pre-purification cold trap; a freezing unit for reducing a temperature of the enrichment cold and hot trap; a vacuum acquisition system for enabling the enrichment and purification device to reach a preset vacuum degree and exhausting other unabsorbed non-target gases out of the enrichment cold and hot trap, having a suction connected to a gas outlet of the enrichment cold and hot trap; a heating unit for heating the enrichment cold and hot trap to completely release the absorbed target gas; and a purification outlet connected to the gas outlet of the enrichment cold and hot trap.

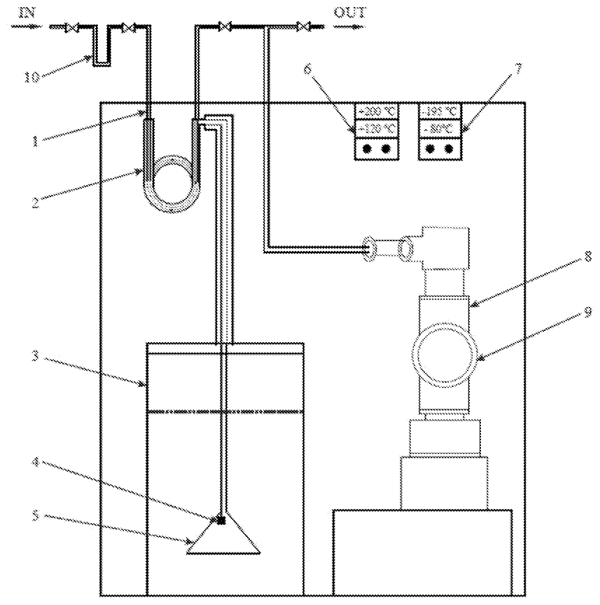

### TECHNICAL FIELD

The invention relates to the technical field of gas analysis instruments, and more particularly, to an enrichment and purification device for a single gas in a mixed gas.

### BACKGROUND OF THE INVENTION

It is a very important analytical method and pretreatment method in the analysis of analytical chemistry, geochemistry and environmental science to extract and purify a gas in a mixed gas of different molecular weights.

In the study of earth science, the analysis of volatiles in a mineral inclusion is of important scientific significance, but with traditional means of analysis, under a vacuum condition and after the mineral is broken, the gases of different molecular weights in the volatiles typically include gaseous H₂O, CO₂, SO₂, NH₃, N₂, O₂, inert gases and the like, leading to difficulty in distinguishing them.

In addition, at present, for the extraction and purification of a gas of a specific molecular weight in a mixed gas, and the preparation of cold liquid for freezing are generally done manually, which is time-consuming and labor-intensive, and it is also difficult to accurately control the freezing temperature of the cold liquid, which in turn affects the accuracy of the analysis.

### SUMMARY OF THE INVENTION

In view of this, an objective of the invention is to provide an enrichment and purification device for a single gas in a mixed gas, so as to realize the extraction and purification of a single gas in a mixed gas with different molecular weights.

In order to achieve the above objective, the invention provides the following technical solutions.

An enrichment and purification device for a single gas in a mixed gas includes:
a pre-purification cold trap capable of freezing and adsorbing part of non-target gases in the mixed gas, the pre-purification cold trap provided with a mixed gas inlet for the mixed gas to enter;
an enrichment cold and hot trap capable of adsorbing the target gas in the mixed gas when frozen, and capable of releasing the adsorbed purified target gas when heated, the enrichment cold and hot trap detachably filled with molecular sieves of different specifications, the gas inlet of the enrichment cold and hot trap connected with the gas outlet of the pre-purification cold trap through a first switching valve;
a freezing unit for reducing the temperature of the enrichment cold and hot trap so that the enrichment cold and hot trap absorbs the target gas;
a vacuum acquisition system for enabling the enrichment and purification device to reach a preset vacuum degree and capable of exhausting other non-target gases in the mixed gas that are not adsorbed out of the vacuum acquisition system of the enrichment cold and hot trap, the suction of the vacuum acquisition system connected with the gas outlet of the enrichment cold and hot trap;
a heating unit for heating the enrichment cold and hot trap to completely release the target gas adsorbed by the enrichment cold and hot trap; and
a purification outlet connected to a gas outlet of the enrichment cold and hot trap through a second switching valve.

Preferably, in the enrichment and purification device described above, the freezing unit includes:
a freezing sleeve sleeved on the enrichment cold and hot trap;
a self-adapting liquid nitrogen tank storing liquid nitrogen, the self-adapting liquid nitrogen tank introducing the liquid nitrogen required for freezing the target gas into the freezing sleeve through a conduit, the conduit extending below the level of the liquid nitrogen of the self-adapting liquid nitrogen tank; and
a heating module for vaporizing the liquid nitrogen in the self-adapting liquid nitrogen tank to generate pressure to introduce the liquid nitrogen into the freezing sleeve.

Preferably, in the enrichment and purification device described above, the freezing unit further includes:
a tapered guide structure provided at the end of the conduit extending into the self-adapting liquid nitrogen tank, the tapered guide structure gradually diverging from the end proximal to the freezing sleeve to the end distant from the freezing sleeve, the heating module disposed in the tapered guide structure.

Preferably, in the enrichment and purification device described above, the freezing sleeve is a polytetrafluoroethylene (Teflon) sleeve, the conduit is a polytetrafluoroethylene tube, and the tapered guide structure is a metal tapered tube.

Preferably, the enrichment and purification device described above further includes:
a first temperature control probe for detecting the freezing temperature of the enrichment cold and hot trap; and
a freezing temperature controller for controlling the amount of liquid nitrogen entering into the freezing sleeve, the freezing temperature controller connected to the first temperature control probe and the heating module.

Preferably, in the above enrichment and purification device described above, the enrichment cold and hot trap is a stainless steel cold and hot trap, and the heating unit is a heating wire provided on the stainless steel cold and hot trap.

Preferably, the enrichment and purification device described above further includes:
a second temperature control probe for detecting the heating temperature of the enrichment cold and hot trap; and
a heating temperature controller for controlling the heating temperature of the heating wire, the heating temperature controller connected to the second temperature control probe and the heating wire.

Preferably, in the enrichment and purification device described above, the vacuum acquisition system includes:
a molecular turbo pump, a suction of the molecular turbo pump connected to the gas outlet of the enrichment cold and hot trap;
a mechanical pump, a suction of the mechanical pump connected to the exhaust of the molecular turbo pump; and
a vacuum gauge for measuring the degree of vacuum of the enrichment and purification device.

Preferably, in the enrichment and purification device descried above, the enrichment cold and hot trap is a spiral tube with a spiral axis.

Preferably, in the enrichment and purification device described above, the pre-purification cold trap is a U-shaped tube with a U-shaped axis.

It may be seen from the above technical solutions that the enrichment and purification device for a single gas in a mixed gas provided by the invention may include: a pre-purification cold trap capable of freezing and adsorbing part of non-target gases in the mixed gas, the pre-purification cold trap provided with a mixed gas inlet for the mixed gas to enter; an enrichment cold and hot trap capable of adsorbing the target gas in the mixed gas when frozen, and capable of releasing the adsorbed purified target gas when heated, the enrichment cold and hot trap detachably filled with molecular sieves of different specifications, the gas inlet of the enrichment cold and hot trap connected with the gas outlet of the pre-purification cold trap through a first switching valve; a freezing unit for reducing the temperature of the enrichment cold and hot trap so that the enrichment cold and hot trap absorbs the target gas; a vacuum acquisition system for enabling the enrichment and purification device to reach a preset vacuum degree and capable of exhausting out of the enrichment cold and hot trap other non-target gases in the mixed gas that are not adsorbed, the suction of the vacuum acquisition system connected with the gas outlet of the enrichment cold and hot trap; a heating unit for heating the enrichment cold and hot trap to completely release the target gas adsorbed by the enrichment cold and hot trap; a purification outlet connected to the gas outlet of the enrichment cold and hot trap through a second switching valve.

It can be applied in the following means: firstly, by the vacuum acquisition system, placing the enrichment and purification device at the preset vacuum degree, and opening the first switching valve and closing the second switching valve; passing the mixed gas through the mixed gas inlet of the pre-purification cold trap to freeze and absorb, by the pre-purification cold trap, part of non-target gases in the mixed gas; then passing the remaining gas into the enrichment cold and hot trap, and reducing, by the freezing unit, the temperature of the enrichment cold and hot trap; and then based on the molecular weight or freezing point of the target gas to be purified, either selecting to adsorb the target gas by the enrichment cold and hot trap itself, or absorbing the target gas through the molecular sieves of different specifications in the enrichment cold and hot trap; after the target gas is completely adsorbed, exhausting, by the vacuum acquisition system, other non-target gases in the mixed gas that are not adsorbed out of the enrichment cold and hot trap; and finally, closing the first switching valve, and opening the second switching valve, and heating, by the heating unit, the enrichment cold and hot trap to completely release the target gas adsorbed by the enrichment cold and hot trap, thereby obtaining a purified target gas at the purification outlet, which may then be sent to next analysis step.

In summary, the enrichment and purification device for a single gas in a mixed gas of the invention can realize the extraction and purification of a single gas in a mixed gas with different molecular weights.

### BRIEF DESCRIPTION OF THE DRAWING

In order to more clearly explain embodiments of the invention or the technical solutions in the prior art, the drawing used in the description of the embodiments or the prior art will be briefly introduced below. It may be apparent for those skilled in the art that the drawing in the following description is some embodiments of the invention, and other drawings may be obtained according to the drawing without creative efforts.

The FIGURE is a schematic structural diagram of an enrichment and purification device for a single gas in a mixed gas according to an embodiment of the invention.

### DETAILED DESCRIPTION OF EMBODIMENTS

Embodiments of the invention provide an enrichment and purification device for a single gas in a mixed gas, so as to realize the extraction and purification of a single gas in a mixed gas with different molecular weights.

In order to make the objectives, technical solutions, and advantages of embodiments of the invention clearer, the technical solutions in the embodiments of the invention will be clearly and completely described with reference to the accompanying drawings in the embodiments of the invention. It is apparent that the described embodiments are a part of embodiments of the invention, but not all of embodiments of the invention. Based on the illustrated embodiments of the invention, all other embodiments obtained by a person of ordinary skill in the art without creative efforts are fall within the protection scope of the invention.

Referring to the FIGURE, an enrichment and purification device for a single gas in a mixed gas provided by an embodiment of the invention includes: a pre-purification cold trap 10 capable of freezing and adsorbing part of non-target gases in the mixed gas, the pre-purification cold trap 10 provided with a mixed gas inlet for the mixed gas to enter; an enrichment cold and hot trap 1 capable of adsorbing the target gas in the mixed gas when frozen, and capable of releasing the adsorbed purified target gas when heated, the enrichment cold and hot trap 1 detachably filled with molecular sieves of different specifications, the gas inlet of the enrichment cold and hot trap 1 connected with the gas outlet of the pre-purification cold trap 10 through a first switching valve; a freezing unit configured (i.e., structured and arranged) for reducing the temperature of the enrichment cold and hot trap 1 so that the enrichment cold and hot trap 1 absorbs the target gas; a vacuum acquisition system configured for enabling the enrichment and purification device to reach a preset vacuum degree and capable of exhausting other non-target gases in the mixed gas that are not adsorbed out of the enrichment cold and hot trap 1, the suction of the vacuum acquisition system connected with the gas outlet of the enrichment cold and hot trap 1; a heating unit configured for heating the enrichment cold and hot trap 1 to completely release the target gas adsorbed by the enrichment cold and hot trap 1; a purification outlet connected to the gas outlet of the enrichment cold and hot trap 1 through a second switching valve.

The enrichment cold and hot trap 1 is used/configured to freeze and enrich the target gas to be analyzed.

The vacuum acquisition system is used to obtain the required vacuum degree in the system, and keep the vacuum of the pipeline and the pre-purification cold trap 10 and the enrichment cold and hot trap 1.

Specifically, the pre-purification cold trap 10 freezes and absorbs part of non-target gases in the mixed gas by a liquid nitrogen cup sleeved thereon. Certainly, depending on the non-target gases to be adsorbed, the liquid nitrogen cup may also be replaced with other freezing devices, such as a carbon dioxide cold liquid device, etc.

It can be applied in the following means: firstly, by the vacuum acquisition system, placing the enrichment and purification device at the preset vacuum degree, and opening the first switching valve and closing the second switching valve; passing the mixed gas through the mixed gas inlet of the pre-purification cold trap 10 to freeze and absorb, by the pre-purification cold trap 10, part of non-target gases in the mixed gas; then passing the remaining gas into the enrichment cold and hot trap 1, and reducing, by the freezing unit, the temperature of the enrichment cold and hot trap 1; and then based on the molecular weight or freezing point of the target gas to be purified, either selecting to adsorb the target gas by the enrichment cold and hot trap 1 itself, or absorbing the target gas through the molecular sieves of different specifications in the enrichment cold and hot trap 1; after the target gas is completely adsorbed, exhausting, by the vacuum acquisition system, other non-target gases in the mixed gas that are not adsorbed out of the enrichment cold and hot trap 1; and finally, closing the first switching valve, and opening the second switching valve, and heating, by the heating unit, the enrichment cold and hot trap 1 to completely release the target gas adsorbed by the enrichment cold and hot trap 1, thereby obtaining a purified target gas at the purification outlet, which may then be sent to next analysis step.

In summary, the enrichment and purification device for a single gas in a mixed gas provided in this embodiment can realize the extraction and purification of a single gas in a mixed gas of different molecular weights. The gases that may be directly frozen, enriched and purified include H₂O, CO₂, SO₂, NH₃, N₂, and O₂, etc. O₂ and N₂ may be frozen, enriched and purified by loading 10A and 5A molecular sieves into the enrichment cold and hot trap 1, respectively.

Preferably, the freezing unit includes a freezing sleeve 2; a self-adapting liquid nitrogen tank 3 storing liquid nitrogen, the self-adapting liquid nitrogen tank 3 introducing the liquid nitrogen required for freezing the target gas into the freezing sleeve 2 through a conduit, the conduit extending below the level of the liquid nitrogen of the self-adapting liquid nitrogen tank 3; a heating module (also referred to as heater) 4 for vaporizing the liquid nitrogen in the self-adapting liquid nitrogen tank 3 to generate pressure to introduce the liquid nitrogen into the freezing sleeve 2.

Specifically, the inner diameter of the freezing sleeve 2 is about 2 mm larger than the outer diameter of the enrichment cold and hot trap 1. The liquid nitrogen in the freezing sleeve 2 is used to cool the enrichment cold and hot trap 1 to a low temperature required for freezing the target gas. The self-adapting liquid nitrogen tank 3 is in a semi-sealed state, and only supplies liquid nitrogen to the freezing sleeve 2 through a conduit.

In this embodiment, the liquid nitrogen in the self-adapting liquid nitrogen tank 3 is vaporized by the heating module 4 to generate pressure to introduce the liquid nitrogen automatically into the freezing sleeve 2 through the conduit, to reduce the temperature of the enrichment cold and hot trap 1 with the freezing sleeve 2, so that the enrichment cold and hot trap 1 reaches the freezing temperature of the target gas. The liquid nitrogen entering into the freezing sleeve 2 may be adjusted according to different target gas, thereby facilitating the control of the freezing temperature of the enrichment cold and hot trap 1.

It may be appreciated that the above-mentioned freezing unit may also be a liquid nitrogen bucket, which achieves the same effect of adjusting the freezing temperature of the enrichment cold and hot trap 1 by controlling the amount of liquid nitrogen in the liquid nitrogen bucket. Certainly, the above liquid nitrogen may also be replaced with another cold liquid, such as a mixed liquid of liquid nitrogen and anhydrous alcohol, or a carbon dioxide cold liquid and the like.

In a further technical solution, the freezing unit further includes a tapered guide structure provided at the end of the conduit extending into the self-adapting liquid nitrogen tank 3, the tapered guide structure gradually diverging from the end proximal to the freezing sleeve 2 to the end distant from the freezing sleeve 2, the heating module 4 disposed in the tapered guide structure. The tapered guide structure is used to gather the pressure generated by the heating module 4 in heating the liquid nitrogen, and push the liquid nitrogen into the freezing sleeve 2 to facilitate the output of the liquid nitrogen. Of course, in the embodiment, the above-mentioned tapered guide structure may also be replaced with a structure with an opening larger than the through hole of the conduit.

In order to prolong the life, the freezing sleeve 2 is a polytetrafluoroethylene sleeve, the conduit is a polytetrafluoroethylene tube, and the tapered guide structure is a metal tapered tube 5. Of course, the above components may also be replaced with other suitable materials.

Preferably, the enrichment and purification device further includes a first temperature control probe configured for detecting the freezing temperature of the enrichment cold and hot trap 1; a freezing temperature controller 7 configured for controlling the amount of liquid nitrogen entering into the freezing sleeve 2, the freezing temperature controller 7 connected to the first temperature control probe and the heating module 4. The first temperature control probe is placed at the enrichment cold and hot trap 1. This embodiment automatically controls the amount of liquid nitrogen entering into the freezing sleeve 2 through the freezing temperature controller 7. When the freezing temperature of the enrichment cold and hot trap 1 detected by the first temperature control probe is higher than the preset target gas freezing temperature, the freezing temperature controller 7 controls the heating unit to heat up. When the freezing temperature of the enrichment cold and hot trap 1 detected by the first temperature control probe is lower than the preset target gas freezing temperature, the freezing temperature controller 7 controls heating unit to reduce the temperature. It saves time and effort, and facilitates accurate control of the freezing temperature of the cold liquid, which improves the accuracy of the analysis. The invention may also manually control the heating temperature of the heating module 4.

To further simplify the structure, the enrichment cold and hot trap 1 is a stainless steel cold and hot trap, and the heating unit is a heating wire provided on the stainless steel cold and hot trap. In this embodiment, the stainless steel cold and hot trap is directly heated by the heating wire, thereby releasing the adsorbed target gas. This structure is relatively simple. Certainly, the above-mentioned enrichment cold and heat trap 1 may also use other media. The heating unit may also have other structures, such as a heater.

The enrichment and purification device further includes a second temperature control probe configured for detecting the heating temperature of the enrichment cold and hot trap 1; a heating temperature controller 6 configured for controlling the heating temperature of the heating wire, the heating temperature controller 6 connected to the second temperature control probe and the heating wire. The second temperature control probe is placed at the enrichment cold and hot trap 1. This embodiment automatically controls the heating temperature of the enrichment cold and hot trap 1 through the heating temperature controller 6. When the heating temperature of the enrichment cold and hot trap 1 detected by the second temperature control is lower than the preset target gas release temperature, the heating temperature controller 6 controls the heating wire to heat up. When the heating temperature of the enrichment cold and hot trap 1 detected by the second temperature control probe is higher than the preset target gas release temperature, the heating temperature controller 6 controls the heating wire to stop heating, which facilitates the accurate control of the heating temperature of the enrichment cold and hot trap 1. The invention may also manually control the heating temperature of the heating wire.

The vacuum acquisition system may includes: a molecular turbo pump 8, the suction of the molecular turbo pump 8 connected to the gas outlet of the enrichment cold and heat trap 1; a mechanical pump, the suction of the mechanical pump connected to the exhaust of the molecular turbo pump 8; a vacuum gauge 9 for measuring the degree of vacuum of the enrichment the purification device. As the structure of the vacuum acquisition system is relatively simple, and certainly may also be replaced with other structures capable of vacuuming, it will not be exemplified in detail in the invention.

In order to ensure the adsorption effect of the enrichment cold and hot trap 1, the enrichment cold and hot trap 1 is a spiral tube having a spiral axis. The adsorption capacity of the enrichment cold and hot trap 1 is relatively large, which ensures the purification effect. Certainly, the enrichment cold and heat trap 1 may also have other shapes, such as a serpentine tube and the like.

Preferably, the pre-purification cold trap 10 is a U-shaped tube with a U-shaped axis, and may also be other shapes, such as a serpentine tube.

There are two main cases when purifying a specific gas from a mixed gas:

In the first case, a gas with a lighter molecular weight or a relatively low freezing point is extracted from the mixed gas. For example, N₂ and O₂ are extracted and purified from a mixed gas containing H₂O, CO₂, N2, and O₂. First, the vacuum acquisition system, including a mechanical pump and a molecular turbo pump 8, is used to place the pipeline of the enrichment and purification device at the required vacuum degree, and the vacuum gauge 9 is used to determine the vacuum degree.

The mixed gas is passed through the pipeline of the enrichment and purification device, where the pre-purification cold trap 10 is provided with a liquid nitrogen cup sleeved thereon. The mixed gas first passes through the pre-purification cold trap 10. As the freezing points of H₂O and CO₂ are 0° C. and −78.5%° C. respectively, and the temperature of liquid nitrogen is −195.8° C., the H₂O and CO₂, etc. therein will be frozen first. Completion of the freezing may be determined by vacuum gauge 9. As the freezing points of N₂ and O₂ are −209.8° C. and −218° C., so they cannot be adsorbed.

The purified mixed gas of N₂ and O₂ enters into the stainless steel cold and hot trap. At the same time, the freezing temperature controller 7 is used to set the freezing temperature as the nitrogen liquefaction temperature. For the adsorption of N₂ and O₂, molecular sieves must be filled in the stainless steel cold and hot trap to adsorb O₂, where a 10A molecular sieve is required to be filled to absorb O₂, and a 5A molecular sieve is required to be filled to absorb N₂. The adsorbed N₂ or O₂ is purified by a molecular turbo pump 8, and the vacuum gauge 9 may be used to detect the purification process. The purified N2 or O2 then uses the heating temperature controller 6 to set the heating temperature as 120° C., at which the target gas adsorbed in the molecular sieve may be completely desorbed. The degree of gas release may be detected using the vacuum gauge 9. In this way, the N₂ or O₂ extracted and purified from the mixed gas may proceed to the next analysis step.

In the second case, a gas with a relatively heavy molecular weight or a relatively high freezing point is extracted from the mixed gas. Taking the above mixed gas as an example, H₂O or CO₂ is extracted and purified therefrom. First, the vacuum acquisition system, including a mechanical pump and a molecular turbo pump 8, is used to place the pipeline of the enrichment and purification device at the required vacuum degree, and the vacuum gauge 9 is used to determine the vacuum degree.

When extracting and purifying CO₂, the mixed gas passes into the pipeline of the enrichment and purification device. Since the freezing points of H₂O and CO₂ are 0° C. and −78.5° C., respectively, H₂O therein is frozen by controlling the temperature of the pre-purification cold trap 10 to set this temperature between the freezing points of H₂O and CO₂. The completion of freezing may be determined by the vacuum gauge 9. The remaining mixed gas is then entered into the stainless steel cold and hot trap, so that CO₂ is adsorbed in the stainless steel cold and hot trap by setting the temperature of the stainless steel cold and hot trap at lower than the CO₂ adsorption temperature, where the CO₂ adsorption temperature is set to −90° C.

When the purified H₂O is extracted and purified, the mixed gas is passed into the pipeline of the enrichment and purification device, while the pre-purification cold trap 10 is kept at a normal temperature. The extraction and purification processes are both performed in a stainless steel cold and hot trap. The freezing temperature controller 7 is used to set the H₂O adsorption temperature lower than the freezing point of H₂O. The H₂O adsorption temperature is set to −15° C. to ensure that only H₂O is adsorbed.

After the purification of the above two gases, the vacuum gauge 9 is used to determine the completion of the adsorption. After the adsorption is completed, the molecular turbine pump 8 is first used to pump away other gases in the pipeline to achieve the purpose of purifying the target gas. The purification process is detected using a vacuum gauge 9. When the gas purification is completed, the heating temperature is set to 120° C. by using the heating temperature controller 6, which may be able to completely desorb the target gas adsorbed in the stainless steel cold and hot trap. The degree of gas release is detected by the vacuum gauge 9. In this way, the H₂O or CO₂ extracted and purified from the mixed gas may proceed to the next analysis step.

The enrichment and purification device for a single gas in a mixed gas provided by the invention may have the following advantages.

1. Broad scope of application. No matter it is a simple mixed gas or a complex mixed gas mixed with different molecular weights, the invention may effectively enrich and purify a gas of a certain molecular weight.

2. High accuracy of analysis. Because the high-vacuum pipeline design of the molecular turbo pump 8 is adopted, and the internal volume of the system pipeline is small, the analysis accuracy may be improved by an order of magnitude compared with the traditional methods.

3. Efficient and simple analysis process. As the integrated module design is adopted, and the freezing and desorption of the system are controlled automatically, the analysis efficiency is greatly improved compared with the traditional methods.

The invention will provide an efficient and feasible instrument analysis platform for the research of earth science and environmental science.

The embodiments in this specification are described in a progressive manner Each embodiment focuses on the differences from other embodiments. Simply refer to each other for the same and similar parts between the embodiments.

The above description of the disclosed embodiments enables those skilled in the art to implement or use the invention. Various modifications to these embodiments will be apparent to those skilled in the art, and the general principles defined herein may be implemented in other embodiments without departing from the spirit or scope of the invention. Therefore, the invention will not be limited to the embodiments shown herein, but should conform to the widest scope consistent with the principles and novel features disclosed herein.

What is claimed is:

1. An enrichment and purification device for a single gas in a mixed gas, comprising:
a pre-purification cold trap configured to freeze and adsorb part of non-target gases in the mixed gas, wherein the pre-purification cold trap is provided with a mixed gas inlet for the mixed gas to enter;
an enrichment cold and hot trap configured to adsorb a target gas in the mixed gas when frozen, and release an adsorbed purified target gas when heated, wherein the enrichment cold and hot trap is detachably filled with molecular sieves of different specifications, a gas inlet of the enrichment cold and hot trap is connected to a gas outlet of the pre-purification cold trap through a first switching valve;
a freezing unit configured to reduce a temperature of the enrichment cold and hot trap so that the enrichment cold and hot trap absorbs the target gas;
a vacuum acquisition system configured to enable the enrichment and purification device to reach a preset vacuum degree and exhaust other non-target gases in the mixed gas that are not adsorbed out of the enrichment cold and hot trap, wherein a suction of the vacuum acquisition system is connected to a gas outlet of the enrichment cold and hot trap;
a heating unit configured to heat the enrichment cold and hot trap to completely release the target gas adsorbed by the enrichment cold and hot trap; and
a purification outlet connected to the gas outlet of the enrichment cold and hot trap through a second switching valve.

2. The enrichment and purification device according to claim 1, wherein the freezing unit comprises:
a freezing sleeve sleeved on the enrichment cold and heat trap;
a self-adapting liquid nitrogen tank storing liquid nitrogen, wherein the self-adapting liquid nitrogen tank is configured to introduce a liquid nitrogen required for freezing the target gas into the freezing sleeve through a conduit, and the conduit extends below a level of the liquid nitrogen of the self-adapting liquid nitrogen tank; and
a heating module configured to vaporize the liquid nitrogen in the self-adapting liquid nitrogen tank to generate pressure to introduce the liquid nitrogen into the freezing sleeve.

3. The enrichment and purification device according to claim 2, wherein the freezing unit further comprises a tapered guide structure provided at an end of the conduit extending into the self-adapting liquid nitrogen tank, the tapered guide structure gradually diverges from an end proximal to the freezing sleeve to another end distant from the freezing sleeve, and the heating module is disposed in the tapered guide structure.

4. The enrichment and purification device according to claim 3, wherein the freezing sleeve is a polytetrafluoroethylene sleeve, the conduit is a polytetrafluoroethylene tube, and the tapered guide structure is a metal tapered tube.

5. The enrichment and purification device according to claim 2, further comprising:
a first temperature control probe configured to detect a freezing temperature of the enrichment cold and hot trap; and
a freezing temperature controller configured to control an amount of the liquid nitrogen entering into the freezing sleeve, wherein the freezing temperature controller is connected to the first temperature control probe and the heating module.

6. The enrichment and purification device according to claim 1, wherein the enrichment cold and hot trap is a stainless steel cold and hot trap, and the heating unit is a heating wire provided on the stainless steel cold and hot trap.

7. The enrichment and purification device according to claim 6, further comprising:
a second temperature control probe configured to detect a heating temperature of the enrichment cold and hot trap; and
a heating temperature controller configured to control a heating temperature of the heating wire, wherein the heating temperature controller is connected to the second temperature control probe and the heating wire.

8. The enrichment and purification device according to claim 1, wherein the vacuum acquisition system comprises:
a molecular turbo pump, wherein a suction of the molecular turbo pump is connected to the gas outlet of the enrichment cold and hot trap;
a mechanical pump, wherein a suction of the mechanical pump is connected to an exhaust of the molecular turbo pump; and
a vacuum gauge configured to measure a degree of vacuum of the enrichment and purification device.

9. The enrichment and purification device according to claim 1, wherein the enrichment cold and hot trap is a spiral tube having a spiral axis.

10. The enrichment and purification device according to claim 1, wherein the pre-purification cold trap is a U-shaped tube with a U-shaped axis.
